(19) United States
(12) Patent Application Publication
(10) Pub. No.: US 2021/0290606 A1
(43) Pub. Date: Sep. 23, 2021

(54) METHOD TO PREVENT AND TREAT OSTEOARTHRITIS BY CALCIUM CHANNEL BLOCKERS, ANGIOTENSIN CONVERTING ENZYME INHIBITORS, AND ANGIOTENSIN RECEPTOR BLOCKERS

(71) Applicant: Assa Weinberg, Los Angeles, CA (US)

(72) Inventor: Assa Weinberg, Los Angeles, CA (US)

(21) Appl. No.: 17/339,569

(22) Filed: Jun. 4, 2021

Related U.S. Application Data
(63) Continuation of application No. PCT/US2019/065071, filed on Dec. 6, 2019.
(60) Provisional application No. 62/776,990, filed on Dec. 7, 2018.
(60) Provisional application No. 62/776,871, filed on Dec. 7, 2018.
(60) Provisional application No. 62/776,683, filed on Dec. 7, 2018.

(51) Int. Cl. A61K 31/4422; A61K 9/00; A61K 31/401; A61P 19/02; A61K 31/4178

(52) U.S. Cl. CPC A61K 31/4422; A61K 9/0014; A61K 9/0019; A61P 19/02; A61K 31/4178; A61K 31/401

(57) ABSTRACT

A method is provided to prevent and to treat Osteoarthritis syndrome by using Calcium Channel Blockers, Angiotensin-Converting Enzyme (ACE) Inhibitors, or Angiotensin Receptor Blockers (ARB), and more particularly, to a method to prevent and treat Osteoarthritis syndrome by using Calcium Channel Blockers, Angiotensin-Converting Enzyme Inhibitors, or Angiotensin Receptor Blockers that are not taken orally, but administered by intra-articular injection into the affected joint or administered by topical application to the skin in a region of the affected joint, so as to increase the capillary network and articular blood supply to the joint or to increase hyaluronic acid production to the articular cartilage, bones, articular ligaments, synovia tissue and other soft tissues.

### INCORPORATION BY REFERENCE TO RELATED APPLICATION

This application is a continuation of and claims benefit of PCT Application No. PCT/US2019/065071, filed Dec. 6, 2019, which claims benefit of U.S. Provisional Application No. 62/776,683, filed Dec. 7, 2018, U.S. Provisional Application No. 62/776,871, filed Dec. 7, 2018, and U.S. Provisional Application No. 62/776,990, filed Dec. 7, 2018. Each of the aforementioned applications is incorporated by reference herein in its entirety, and each is hereby expressly made a part of this specification.

### FIELD OF THE INVENTION

A method is provided to prevent and to treat Osteoarthritis syndrome by using Calcium Channel Blockers, Angiotensin-Converting Enzyme (ACE) Inhibitors, or Angiotensin Receptor Blockers (ARB), and more particularly, to a method to prevent and treat Osteoarthritis syndrome by using Calcium Channel Blockers, Angiotensin-Converting Enzyme Inhibitors, or Angiotensin Receptor Blockers that are not taken orally, but administered by intra-articular injection into the affected joint or administered by topical application to the skin in a region of the affected joint, so as to increase the capillary network and articular blood supply to the joint or to increase hyaluronic acid production to the articular cartilage, bones, articular ligaments, synovia tissue and other soft tissues.

### BACKGROUND OF THE INVENTION

Osteoarthritis surpasses all other human diseases in two fields. It tops the list of human diseases that cause pain. It is the leading disease in the world that causes disability. Osteoarthritis is the most common among diseases that attacks human joints. Osteoarthritis inflicts substantial morbidity at all ages but in the elderly it is associated with significant increase in mortality.

The three most common symptoms for the disease are: pain, stiffness, and reduced joint mobility. The disease is the world leader among chronic diseases that relentlessly progresses slowly.

The world prevalence of osteoarthritis is rising rapidly. The number of patients reported to be afflicted with osteoarthritis has increased by 40 percent between 1990 to 2016. Worldwide, the disease affects 240 million people. It affects mostly the elderly. Women are more affected the men.

The most affected joints are the weight-bearing lower extremity joints, followed by the hands and the spine joints. Of the 30 million people in the US that are diagnosed with osteoarthritis, nearly half have osteoarthritis of the knee. 14 million people have x-ray confirmed symptomatic osteoarthritis of the knee.

The disease does not spare the young. It affects 5 percent of those aged below 18.7 percent of all adults are below 25. This number nearly triples and rises to 19 percent for those above the age of 65, one out of six. The lifetime risk of symptomatic osteoarthritis of the knee is 40 percent for men, and 47 percent of women.

Over the last 20 years, a major scientific effort was directed to uncover the causes and discover a treatment for osteoarthritis. Considerable knowledge was gained in three different fields: 1. New biological pathways that lead to joint destruction were discovered; 2. Risk factors that affect the prevalence and the progression of the disease were identified; and 3. New treatment modalities that reduce pain and increase osteoarthritic joints mobility surfaced.

Laboratory work on genetically modified animals and human joints led to the dismissal of the 20th century concept that osteoarthritis is a simple wear and tear disease that the destruction of the matrix of the articular cartilage is purely due to mechanical forces.

Instead, osteoarthritis was found to be an inflammatory disease that affects not only the cartilage but affects the entire joint structures: bones, ligaments, synovial membrane and other soft tissues.

Four separate biological pathways with complex and elaborate interactions are now known to drive the osteoarthritic progressive destructive process. These are: 1. New and specific pro-inflammatory cytokines that promote the degrading of the elements of joints structure process were identified. 2. Bio-mechanical forces were revealed that involve a repetitive use of abnormal joint mechanics, leading to pathological release of pro-inflammatories, cytokines, and destructive proteolytic enzymes. 3. Cartilage digesting enzymes that degrade the articular matrix and other joint structural and architectural components were isolated. 4. Non-inflammatory, destructive pathways that inhibit joint's ability to repair damaged structures. These include the discovery of epigenetic forces that modify the normal transcription program of joint genes.

A considerable body of knowledge was gained by the search for risk factors that affect the prevalence and the clinical progression of the osteoarthritis syndrome.

A systematic review of thousands of published scientific research work, and the use of meta-analyses lead to the discovery of 8 risk factors with high statistical power. They are: Aging the disease is clearly age related. Both the incidence and the progression of osteoarthritis are dominated by age, but aging and osteoarthritis are distinct processes; Gender—the disease affects more women than men; Genetics—genetics is responsible for 40 percent of osteoarthritis cases and dominate the osteoarthritis that appears in adolescence; Mutation in GDF 5—a gene that codes for growth and differentiation factor was found by a genome-wide association study to be common in patients with osteoarthritis; Anatomical design—certain inborn alignments of the femur and the tibia knee bones, in particular the varus angulation (bow legs), and to a lesser extent, the angulation of the valgus alignment are prone to lead to osteoarthritis. None of these factors can be subject or modified by any treatment.

Four modifiable risk factors that affect the prevalence and the severity of this disease were identified as well. Direct medical intervention on each one was found to delay the onset of osteoarthritis, or confer a protective effect. Those are: 1. Morbid obesity; 2. Smoking; 3. Bone density; 4. Physical activity.

Unfortunately, the same medical manipulation that reduce the incidence and severity of osteoarthritis, increases the mortality of these patients by other diseases.

Morbid obesity, (BMI above 30); and even BMI above 25 but below 30, increased 6 fold the incidence of osteoarthritis, but recreational walking or jogging, which are known to reduce mortality from cardiovascular diseases had no protective effect, and increases the severity and prevalence of osteoarthritis. Reduction in walking has the opposite effect.

The relation of smoking to osteoarthritis is a subject of debate. Meta-analyses from 2011 of 48 observational studies, of 537730 participants found that smoking increases the incidence of osteoarthritis. A more recent meta-analysis of 38 observational studies from 2016 that included 481,744 participants revealed that smoking which increases mortality by cancer and cardiovascular disease has inverse effect (protective) on this disease.

A higher bone mass (bone density) which protects against bone fractures, especially of the hip, and decreases overall mortality, is associated with a significant increase in three elements of the disease: radiographic incidence of symptomatic osteoarthritis, the use of NSAIDs, and joint replacements. Increase in bone density can cause an increase in cartilage loss even in non-weight-bearing joints.

A 2016 meta-analysis of 46 studies found an increase in osteoarthritis incidence by participating in sports and intense physical activity. Using the Framingham cohort data, no benefits were shown by recreational walking or jogging on the incidence of radiological osteoarthritic knees, even in subjects with BMI less than 30.

The above modifiable risk factors were found to affect disease occurrence but only a modest impact on disease outcome. No risk factor elimination can reverse the osteoarthritic process and convert the cartilage to its original structure.

An impressive increase in treatment modalities appeared in the last 20 years. At least 15 different non-surgical modes of treatment were published since 2003. During that period, each of these treatment modality was subject to multiple publications in diverse medical journals by diverse clinical investigators groups. Nearly all were subject to meta-analysis scrutiny. Amazingly and unfortunately, 10 of the 12 nonsurgical treatments that are now extensively practiced in US for the managements of osteoarthritis, failed to produce superior results to placebo. These include: Insoles and bracing—A meta-analysis of 13 studies that included 1356 patients found no change in symptoms or function compared to placebo. Similar results were found by comparing insoles to braces; Glucosamine sulfate—While an industry-sponsored study of 604 patients found a statistical significant reduction in pain and increase in function with the use of glucosamine sulfate, a meta-analysis of 25 randomized placebo controlled clinical trials with 3458 patients failed to find any significant effects.

Another meta-analysis of 6 published clinical trials addressed the issues of prevention and progression of osteoarthritis by glucosamine sulfate. The trials that included 1501 participants found no preventive effect after one year. While a small statistical effect did surface after 3 years, the practical proven benefit was clinically irrelevant. A meta-analysis of 20 randomized clinical studies that included 3846 patients on pain and function with the use of chondroitin sulfate found only a negligible effect (0.003 percent). A multicenter randomized placebo controlled trial that included 164 patients on pain and function with the use of a Glucosamine and chondroitin combination found that the placebo group had a superior effect on pain relief over the combination treatment group at the end of the six months trial. A 2016 meta-analysis of 22 clinical trials that included 8275 patients found no clinically observable difference between hydromorphone, morphine, oxycodone and the low potency narcotic, tramadol, after one year. Intra-articular hyaluronic injections: A meta-analysis of 19 double blind placebo controlled randomized superiority studies did not find any clinically relevant effect for Intra-articular hyaluronic injections over placebo. Even though multiple published clinical trials found significant clinical benefits with the use of platelet rich plasma, the majority of these publications fail to satisfy a rigorous statistical design and insufficient blinding that compromised the double blind aspect on these trials. No solid, randomized, double blind clinical trial of the use of PRP for osteoarthritis, that passes the scrutiny of the NIH statistical standards, has shown superiority over placebo. A meta-analysis of 74 randomized clinical trials with 58556 patients, demonstrated Tylenol did not have a clinically relevant effect. A meta-analysis of 18 randomized clinical trials that included 813 patients could not confirm any clinically significant beneficial effect on pain relief for Transcutaneous nerve stimulation (TENS). A meta-analysis of 11 clinical trials that compared acupuncture to sham acupuncture treatment of knee osteoarthritis found a short-term statistical difference between the two in pain reduction after 10 sessions. The clinical benefits were themselves irrelevant.

Even FDA approved pharmacological drugs for knee osteoarthritis, such as oral NSAIDs, topical NSAIDs, and capsaicin, affects only pain but not the progression of the disease.

These data highlights the substantial limitations of the current prevention methods and the limited and insignificant role of current treatment modalities of osteoarthritis in reversing the course of this disease.

### SUMMARY OF THE INVENTION

Currently, No FDA approved drug for the treatment of osteoarthritis is known to block the four pathological disease processes that lead to osteoarthritic joint destruction. There currently are no FDA approved medication known to reverse the clinical course of the disease. These facts were underlined by a recent review of 800 published treatment clinical trials of osteoarthritis by the University of North Carolina, which concluded that there is an enormous need to identify a nouvelle approach to reduce the prevalence of this disabling disease.

These data highlight the need for a new method of prevention and treatment.

A method of administering, by intra-articular injection, a pharmaceutical preparation, in an effective amount, of a Calcium Channel Blocker, ACE Inhibitor, or Angiotensin Receptor Blocker, directly to the knee, where the symptoms are known to have formed, is provided to prevent osteoarthritis of the knee symptoms.

Additionally, a method is provided of administering by intra-articular injection, a pharmacological composition in an effective amount, of a Calcium Channel Blocker, ACE Inhibitor, or Angiotensin Receptor Blocker, directly to the knee to treat the symptoms of osteoarthritis of the knee.

Pharmacological preparation as used herein is a pharmacological preparation according to the invention, composed of, but not limited to, a Calcium Channel Blocker, ACE Inhibitor, or Angiotensin Receptor Blocker and a suitable nontoxic pharmacological carrier.

Effective amount as used herein is an amount of the pharmaceutical composition, which is effective for treating osteoarthritis. An amount of a Calcium Channel Blocker, ACE Inhibitor, or Angiotensin Receptor Blocker administered through the epidermis to the knee space.

Contact Calcium Channel Blockers ACE Inhibitor, and Angiotensin Receptor Blockers are a new pharmaceutical class of medications that increase the capillary network and blood supply to the articular cartilage, bones, articular ligaments, the synovial membrane and other soft tissues, to prevent the process and symptoms of osteoarthritis of the knee.

They also increase the production of hyaluronic acid by hyaluronic producing cells.

For the foregoing reasons, there is a need for a method to prevent the articular damage and associated symptoms caused by osteoarthritis by increasing the capillary network and articular blood supply and increase hyaluronic acid production to the articular cartilage, bones, articular ligaments, synovia tissue and other soft tissues.

For the foregoing reasons, there is a need for a method to treat the articular damage and associated symptoms caused by osteoarthritis by increasing the capillary network and articular blood supply and increase hyaluronic acid production to the articular cartilage, bones, articular ligaments, synovial tissue and other subcutaneous soft tissue.

The present invention is directed to a method for preventing osteoarthritis by direct intra-articular injection to the knee space, of a pharmaceutical preparation of Calcium Channel Blockers, ACE Inhibitors, or Angiotensin Receptor Blockers to prevent the formation and symptomatology of knee osteoarthritis.

By osteoarthritis as used herein, it is meant any process in the knee that cause the symptoms of osteoarthritis syndrome.

In addition, the present invention is directed for the treatment of patients with osteoarthritis of any gender caused by or aggravated by but not limited to: injury, obesity, genetic factors, anatomical mal-alignments, smoking, lack of, or insufficient physical activity.

In one method of the invention the Calcium Channel Blockers, ACE Inhibitors, or Angiotensin Receptor Blockers may be administered directly to the articular space before symptoms form to prevent osteoarthritis of the knee. For example, in elderly men or women with knee pain, but without the radiological findings of osteoarthritis.

In another embodiment, the Calcium Channel Blockers, ACE Inhibitors, or Angiotensin Receptor Blockers may be administered directly to the articular cavity to treat and heal the symptoms of osteoarthritis syndrome.

The Calcium Channel Blockers, ACE Inhibitors, or Angiotensin Receptor Blockers may be administered even after the osteoarthritis syndrome has dissipated and the articular cartilage has healed to prevent the recurrence of osteoarthritis syndrome.

Calcium Channel Blockers, ACE Inhibitors, and Angiotensin Receptor Blockers are a class of pharmaceutical drugs that dilate the arteriolar system. They also increase the capillary blood supply and production of hyaluronic acid different tissues.

Currently, there are 20 pharmaceutical patented Angiotensin Receptor Blocker drugs that use this property to treat hypertension, angina pectoris and cardiac arrhythmia. The clinical indication of Angiotensin Receptor Blockers is therefore limited to the field of cardiovascular diseases. Currently, there are over 60 pharmaceutical patented Calcium Channel Blocker drugs that use this property to treat hypertension, angina pectoris and cardiac arrhythmia. The clinical indication of Calcium Channel Blockers is therefore limited to the field of cardiovascular diseases.

Calcium Channel Blockers, ACE Inhibitors, and Angiotensin Receptor Blockers were extensively studied, but their ability to prevent and treat the osteoarthritis syndrome remains unknown.

The present invention introduces the use of intra-articular Calcium Channel Blockers, ACE Inhibitors, or Angiotensin Receptor Blockers to joint space, in particular, the knee joint space. The direct contact of Calcium Channel Blockers, ACE Inhibitors, or Angiotensin Receptor Blockers with the knee connective tissue increases the production of capillary network and augments the blood supply to the intra-articular structures of the knee. They also increase the production of hyaluronic acid by hyaluronic producing cells.

This new class of pharmaceutical drugs may be used for the prevention and treatment of osteoarthritis of the knee or other syndromes associated with cartilage damage.

No trial of Calcium Channel Blockers, ACE Inhibitors, or Angiotensin Receptor Blockers for the prevention and treatment of osteoarthritis of the knee syndrome was ever published.

### DETAILED DESCRIPTION OF THE INVENTION

The following discussion addresses a number of embodiments and applications of the present disclosure. The beneficial features of the present disclosure will be evident from the described embodiments. It is to be understood that the present disclosure is not limited to such specific applications and that numerous implementations of the present disclosure may be realized. All references to patents, patent applications, and non-patent publications mentioned in the specification are hereby incorporated by reference, in their entireties.

Contact neo-vasodilators are a new class of medication. This invention describes the use of contact neo-vasodilators such as Valsartan, a known Angiotensin Receptor Blocker or Enalapril, a known ACE Inhibitor, or calcium channel blockers used in the treatment of hypertension and congestive heart failure, for the treatment of osteoarthritis syndrome.

ACE Inhibitors such as enalapril, benazepril, lisinopril, ramipril, or fosinopril or Angiotensin Receptor Blockers such as Valsartan, telmisartan, olmesartan, losartan, irbesartan, candesartan and azilsartan, when administered topically or by intra-articular injection, are very effective drugs for the treatment of osteoarthritis, particularly of the knee.

Valsartan, or other Angiotensin Receptor Blockers, or Enalapril, or other ACE Inhibitors are drugs which previously may have been used in treatment of high blood pressure and or congestive heart failure, when applied in a pharmacological composition in an effective amount by topical application to the area of the joint or by intra-articular injection are effective drugs for the treatment of osteoarthritis.

Angiotensin Receptor Blockers such as Valsartan, telmisartan, olmesartan, losartan, irbesartan, candesartan and azilsartan, when applied in a pharmacological composition in an effective amount by topical application to the area of the joint or by intra-articular injection are effective drugs for the treatment of osteoarthritis.

Pharmacological composition as used herein is a pharmaceutical preparation according to the invention, composed but not limited to ACE Inhibitors, Calcium Channel Blockers, or Angiotensin Receptor Blockers and a suitable non-toxic pharmaceutical carrier. Effective amount as used herein is an amount of the pharmaceutical composition of Calcium Channel Inhibitors, ACE Inhibitors, or Angiotensin Receptor Blockers that is effective for treating the osteoarthritis syndrome. An amount of Calcium Channel Inhibitors, ACE Inhibitors, or Angiotensin Receptor Blockers that is suitable for topical administration to the skin in the region of the joint or by intra-articular injection to the area of the joint.

A method is provided of applying a pharmaceutical preparation in an effective amount of one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), topically or by intra-articular injection to treat osteoarthritis syndrome.

The pharmacological preparation can comprise a calcium channel blocker. The calcium channel blocker can be in a suitable nontoxic pharmacological carrier.

The pharmacological preparation can comprise an ACE inhibitor. The ACE inhibitor can be in a suitable nontoxic pharmacological carrier.

The pharmacological preparation can comprise an angiotensin receptor blocker. The angiotensin receptor blocker can be in a suitable nontoxic pharmacological carrier.

An effective amount for treatment of osteoarthritis syndrome is administered. An amount of calcium channel blocker that is suitable for treatment by topical administration or intra-articular injection to treat osteoarthritis syndrome is administered.

An effective amount for treatment of osteoarthritis syndrome is administered. An amount of ACE inhibitor that is suitable for treatment by topical administration or intra-articular injection to treat osteoarthritis syndrome is administered.

An effective amount for treatment of osteoarthritis syndrome is administered. An amount of angiotensin receptor blocker that is suitable for treatment by topical administration or intra-articular injection to treat osteoarthritis syndrome is administered.

Contact vasodilators (e.g., calcium channel blockers, ACE inhibitors, angiotensin receptor blockers, nitrates, alpha blockers, beta blockers, hydralazine, and/or angiotensin receptor-neprilysin inhibitors) are a new class of pharmaceutical medications that increase blood supply, which produces biological changes.

In the case of treatment or prevention of osteoarthritis, these changes can include one or more of increasing the blood supply to the joint, increase the capillary network and articular blood supply and increase hyaluronic acid production to the articular cartilage, bones, articular ligaments, synovia tissue and other soft tissues.

Calcium channel blockers are a new class of pharmaceutical drugs that disrupt the entry of calcium molecules through the L type voltage operated channels to cardiac muscle and blood vessels cells. The blockage of calcium entry causes the relief of arterial spasm.

Currently there are 70 pharmaceutical patented calcium channel blocker drugs that use this property to treat hypertension, angina pectoris and cardiac arrhythmia. The clinical indication for the therapeutic use of calcium channel blockers was therefore limited, until now, to the field of cardiovascular diseases only.

Calcium channel blockers were extensively studied but their ability to prevent and or to treat osteoarthritis remained heretofore unknown.

Accordingly, new uses are provided of contact-applied calcium channel blockers for topical application to the skin in the area of the joint or by intra-articular injection into the affected joint. The new use may be used for the treatment of osteoarthritis syndrome. No trial of topical calcium channel blockers for the prevention or treatment of osteoarthritis syndrome has heretofore been published.

Contact calcium channel blockers are a part of contact-vasodilators, a new class of medication. The use is provided of contact neo-vasodilators such as Nifedipine, a known calcium channel blocker used in the treatment of hypertension, for the treatment of osteoarthritis syndrome.

Nifedipine, Amlodipine, Felodipine, Isradipine, Nicardipine, Nisoldipine and Clevidipine are in a class of dihydropyridines calcium channel blockers. Verapamil and Diltiazem are non-dihydropyridines calcium channel blockers. When applied by contact these are very effective drugs for the treatment of osteoarthritis syndrome.

Inhibitors of angiotensin converting enzyme (ACE) can be employed as vasodilators. Angiotensin II is a chemical produced by the body that primarily circulates in the blood. It causes the muscles surrounding blood vessels to contract, thereby narrowing the vessels. Angiotensin II is formed from angiotensin I in the blood by the enzyme angiotensin converting enzyme (ACE). Angiotensin I in the blood is itself formed from angiotensinogen, a protein produced by the liver and released into the blood. Angiotensin converting enzyme inhibitors (ACE inhibitors) are medications that slow (inhibit) the activity of the enzyme ACE, which decreases the production of angiotensin II. As a result, blood vessels enlarge or dilate. ACE inhibitors include, but are not limited to benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik).

Angiotensin II receptor blockers (ARBs) help relax the blood vessels. Angiotensin II receptor blockers block the action of angiotensin II, allowing blood vessels to dilate. Angiotensin receptor blockers include, but are not limited to: azilsartan (Edarbi), candesartan (Atacand), eprosartan, irbesartan (Avapro), losartan (Cozaar), olmesartan (Benicar), telmisartan (Micardis), and valsartan (Diovan).

Other vasodilators are known in the art. These include, but are not limited to nitrates (nitroglycerin, isosorbide mononitrate and isosorbide dinitrate), Alpha blockers (doxazosin (Cardura), prazosin (Minipress), terazosin), Beta blockers (Acebutolol (Sectral), Atenolol (Tenormin), Bisoprolol fumarate (Zebeta), Carvedilol (Coreg)—Combined alpha/beta blocker, Esmilol (Brevibloc), Labetalol (Trandate, Normodyne)—Combined alpha/beta blocker, Metoprolol tartrate (Lopressor) and metoprolol succinate (Toprol-XL), Nadolol (Corgard), Nebivolol (Bystolic), Penbutolol sulfate (Levatol), Propranolol (Inderal), Sotalol (Betapace), HCTZ and bisoprolol (Ziac) is a beta blocker plus diuretic), Hydralazine, and angiotensin receptor-neprilysin inhibitors (ARNi) (Entresto, sacubitril/valsartan).

### Conditions Amenable to Treatment or Prevention

Compositions and methods are provided for the prevention or treatment of osteoarthritis syndrome, particularly in the knee.

Application of vasodilators (e.g., calcium channel blockers, ACE inhibitors, angiotensin receptor blockers, nitrates, alpha blockers, beta blockers, hydralazine, and/or angiotensin receptor-neprilysin inhibitors), such as Nifedipine or other calcium channel blockers, which previously may have been used in the treatment of high blood pressure, in a pharmacological composition, in an effective amount, in a contact form, such as, but not limited to an oil, liquid preparation or suspension, to the skin or for administration by intra-articular injection, can be employed to trea the symptoms of osteoarthritis syndrome.

Pharmacological compositions of the embodiments include but are not limited to one or more vasodilators (e.g., calcium channel blockers, ACE inhibitors, angiotensin receptor blockers, nitrates, alpha blockers, beta blockers, hydralazine, and/or angiotensin receptor-neprilysin inhibitors) and a suitable non toxic pharmaceutical carrier. The pharmaceutical composition in administered in an amount effective for treating osteoarthritis syndrome, e.g., an amount suitable for treatment by topical application or intra-articular injection.

Osteoarthritis, associated symptoms, and treatment thereof, are described in the following references, each of which is incorporated by reference herein in its entirety and each of which is hereby made a part of this specification: Da Costa B R, Nüesch E, Kasteler R, et al. Oral or transdermal opioids for osteoarthritis of the knee or hip. Cochrane Database Syst Rev 2014; CD003115; Valdes A M, Spector T D. Genetic epidemiology of hip and knee osteoarthritis. Nat Rev Rheumatol 2011; 7:23; Osteoarthritis: national clinical guideline for care and management in adults. In: Conditions. NCCfC (Ed), Royal College of Physicians, London 2008; Zhang Y, Jordan J M. Epidemiology of osteoarthritis. Clin Geriatr Med 2010; 26:355; https://www.oarsi.org/sites/default/files/docs (Accessed on Oct. 30, 2018); http://www.who.int/chp/topics/rheumatic/en/ (Accessed on Oct. 30, 2018); Vina E R, Kwoh C K. Epidemiology of osteoarthritis: literature update. Curr Opin Rheumatol 2018; 30:160; Murphy L, Schwartz T A, Helmick C G, et al. Lifetime risk of symptomatic knee osteoarthritis. Arthritis Rheum 2008; 59:1207; Lane N E, Brandt K, Hawker G, et al. OARSI-FDA initiative: defining the disease state of osteoarthritis. Osteoarthritis Cartilage 2011; 19:478; Loeser R F, Goldring S R, Scanzello C R, Goldring M B. Osteoarthritis: a disease of the joint as an organ. Arthritis Rheum 2012; 64:1697; Liu-Bryan R, Terkeltaub R. Emerging regulators of the inflammatory process in osteoarthritis. Nat Rev Rheumatol 2015; 11:35; Andriacchi T P, Favre J. The nature of in vivo mechanical signals that influence cartilage health and progression to knee osteoarthritis. Curr Rheumatol Rep 2014; 16:463; Troeberg L, Nagase H. Proteases involved in cartilage matrix degradation in osteoarthritis. Biochim Biophys Acta 2012; 1824:133; Liu Q, Zhang X, Dai L, et al. Long noncoding RNA related to cartilage injury promotes chondrocyte extracellular matrix degradation in osteoarthritis. Arthritis Rheumatol 2014; 66:969; Blagojevic M, Jinks C, Jeffery A, Jordan K P. Risk factors for onset of osteoarthritis of the knee in older adults: a systematic review and meta-analysis. Osteoarthritis Cartilage 2010; 18:24; Loeser R F, Collins J A, Diekman B O. Ageing and the pathogenesis of osteoarthritis. Nat Rev Rheumatol 2016; 12:412; Hussain S M, Cicuttini F M, Bell R J, et al. Incidence of total knee and hip replacement for osteoarthritis in relation to circulating sex steroid hormone concentrations in women. Arthritis Rheumatol 2014; 66:2144; Moisio K, Chang A, Eckstein F, et al. Varus-valgus alignment: reduced risk of subsequent cartilage loss in the less loaded compartment. Arthritis Rheum 2011; 63:1002; Oliveria S A, Felson D T, Cirillo P A, et al. Body weight, body mass index, and incident symptomatic osteoarthritis of the hand, hip, and knee. Epidemiology 1999; 10:161; Chaganti R K, Lane N E. Risk factors for incident osteoarthritis of the hip and knee. Curr Rev Musculoskelet Med 2011; 4:99; Hui M, Doherty M, Zhang W. Does smoking protect against osteoarthritis? Meta-analysis of observational studies. Ann Rheum Dis 2011; 70:1231; Kong L, Wang L, Meng F, et al. Association between smoking and risk of knee osteoarthritis: A systematic review and meta-analysis. Osteoarthritis Cartilage 2017; 25:809; Hardcastle S A, Dieppe P, Gregson C L, et al. Osteoarthritis and bone mineral density: Are strong bones bad for joints? Bonekey Rep 2015; 4:624; Gregson C L, Hardcastle S A, Murphy A, et al. High Bone Mass is associated with bone-forming features of osteoarthritis in non-weight bearing joints independent of body mass index. Bone 2017; 97:306; Tran G, Smith T O, Grice A, et al. Does sports participation (including level of performance and previous injury) increase risk of osteoarthritis? A systematic review and meta-analysis. Br J Sports Med 2016; 50:1459; Duivenvoorden T, Brouwer R W, van Raaij T M, et al. Braces and orthoses for treating osteoarthritis of the knee. Cochrane Database Syst Rev 2015; :CD004020; Wu D, Huang Y, Gu Y, Fan W. Efficacies of different preparations of glucosamine for the treatment of osteoarthritis: a meta-analysis of randomised, double-blind, placebo-controlled trials. Int J Clin Pract 2013; 67:585; Lee Y H, Woo J H, Choi S J, et al. Effect of glucosamine or chondroitin sulfate on the osteoarthritis progression: a meta-analysis. Rheumatol Int 2010; 30:357; Reichenbach S, Sterchi R, Scherer M, et al. Meta-analysis: chondroitin for osteoarthritis of the knee or hip. Ann Intern Med 2007; 146:580; Zhang W, Nuki G, Moskowitz R W, et al. OARSI recommendations for the management of hip and knee osteoarthritis: part III: Changes in evidence following systematic cumulative update of research published through January 2009. Osteoarthritis Cartilage 2010; 18:476; Krebs E E, Gravely A, Nugent S, et al. Effect of Opioid vs Nonopioid Medications on Pain-Related Function in Patients With Chronic Back Pain or Hip or Knee Osteoarthritis Pain: The SPACE Randomized Clinical Trial. JAMA 2018; 319:872; Jevsevar D, Donnelly P, Brown G A, Cummins D S. Viscosupplementation for Osteoarthritis of the Knee: A Systematic Review of the Evidence. J Bone Joint Surg Am 2015; 97:2047; Da Costa B R, Reichenbach S, Keller N, et al. Effectiveness of non-steroidal anti-inflammatory drugs for the treatment of pain in knee and hip osteoarthritis: a network meta-analysis. Lancet 2016; 387:2093; Rutjes A W, Nüesch E, Sterchi R, et al. Transcutaneous electrostimulation for osteoarthritis of the knee. Cochrane Database Syst Rev 2009; CD002823; Manheimer E, Linde K, Lao L, et al. Meta-analysis: acupuncture for osteoarthritis of the knee. Ann Intern Med 2007; 146:868; Da Costa B R, Reichenbach S, Keller N, et al. Effectiveness of non-steroidal anti-inflammatory drugs for the treatment of pain in knee and hip osteoarthritis: a network meta-analysis. Lancet 2017; 390:e21; Deny S, Conaghan P, Da Silva J A, et al. Topical NSAIDs for chronic musculoskeletal pain in adults. Cochrane Database Syst Rev 2016; 4:CD007400; De Silva V, El-Metwally A, Ernst E, et al. Evidence for the efficacy of complementary and alternative medicines in the management of osteoarthritis: a systematic review. Rheumatology (Oxford) 2011; 50:911; Godfraind, Theophile, Discovery and development of Calcium Channel Blockers, Frontiers In Pharmacology, May, 2017, Vol 8, article 286; Williams, B., Drug discovery in renin-angiotensin system intervention: past and future. Therapeutic Advances in Cardiovascular Disease, 2016 June; 10(3): 118-25.

Compositions including one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), optionally in combination with conventional therapies, and associated methods for treatment of osteoarthritis syndrome and related symptoms are provided.

Some embodiments relate to a pharmaceutical composition and method of treatment using the pharmaceutical composition, wherein the pharmaceutical composition comprises at least one calcium channel blocker, for example, a calcium channel blocker selected from the group consisting of amlodipine (Norvasc), diltiazem (Cardizem L A, Tiazac), felodipine (Plendil), isradipine (Dynacirc), nifedipine (Adalat, Procardia), nicardipine (Cardene), nimodipine (Nimotop), nisoldipine (Sular), verapamil (Covera-H S, Verelan P M, Calan), verapamil, diltiazem and nicardipine (Cardene I V). Some embodiments relate to a pharmaceutical composition and method of treatment using the pharmaceutical composition, wherein the pharmaceutical composition comprises at least one ACE inhibitors, for example at least one ACE inhibitor selected from the group consisting of benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik). Some embodiments relate to a pharmaceutical composition and method of treatment using the pharmaceutical composition, wherein the pharmaceutical composition comprises at least one angiotensin receptor blocker, for example at least one angiotensin receptor blocker selected from the group consisting of azilsartan (Edarbi), candesartan (Atacand), eprosartan, irbesartan (Avapro), losartan (Cozaar), olmesartan (Benicar), telmisartan (Micardis), and valsartan (Diovan). In certain embodiments, the pharmaceutical composition is in a form suitable for contact administration, e.g., to skin in the area of an affected joint or by intra-articular administration, however other routes of administration are also considered that involve contact of the vasodilator to the tissue to be treated.

The pharmaceutical compositions for treatment of osteoarthritis syndrome can further comprise other pharmaceutically active ingredients. These can include drugs to control pain, for example, nonsteroidal anti-inflammatory drugs such as ibuprofen or naproxen sodium, topical anesthetics such as lidocaine, a drug to fight infections (e.g., antibiotic, antiviral, or antifungal agents). The treatment can be administered in conjunction with other therapies, e.g., the conventional therapies for osteoarthritis syndrome as described elsewhere herein.

The use of topical vasodilators (e.g., calcium channel blockers, ACE inhibitors, angiotensin receptor blockers, nitrates, alpha blockers, beta blockers, hydralazine, and/or angiotensin receptor-neprilysin inhibitors) for treatment of osteoarthritis syndrome is a new class of drugs. The new class may be used for osteoarthritis syndrome, or to enhance efficacy of conventional osteoarthritis syndrome drugs.

In one method of the vasodilator may be applied directly to the skin in the area of the affected joint.

In another embodiment, the vasodilator may be injected directly into the joint to treat osteoarthritis syndrome. The vasodilator may be applied even after the osteoarthritis syndrome has been ameliorated to prevent recurrence of osteoarthritis syndrome.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, and phosphates.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, tocopherols, carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more pharmacologically active ingredients (e.g. vasodilators) disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles, and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

It is to be understood that where compounds disclosed herein (e.g., calcium channel blockers, ACE inhibitors, angiotensin receptor blockers, nitrates, alpha blockers, beta blockers, hydralazine, and/or angiotensin receptor-neprilysin inhibitors) have unfilled valencies, then the valencies are to be filled with hydrogens or isotopes thereof, e.g., hydrogen-1 (protium) and hydrogen-2 (deuterium).

It is understood that the compounds described herein (e.g., calcium channel blockers, ACE inhibitors, angiotensin receptor blockers, nitrates, alpha blockers, beta blockers, hydralazine, and/or angiotensin receptor-neprilysin inhibitors) can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein may include crystalline forms (also known as polymorphs, which include the different crystal packing arrangements of the same elemental composition of a compound), amorphous phases, salts, solvates, and hydrates, e.g., of vasodilators. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein (e.g., vasodilators) may exist in unsolvated as well as solvated forms. In general, the solvated forms are considered equivalent to the unsolvated forms for the purposes of the compounds and methods provided herein.

Where a range of values is provided, it is understood that the upper and lower limit, and any intervening value between the upper and lower limit of the range is included.

Any percentages, ratios or other quantities referred to herein are on a weight basis, unless otherwise indicated.

### Pharmaceutical Compositions

The vasodilators (e.g., calcium channel blockers, ACE inhibitors, angiotensin receptor blockers, nitrates, alpha blockers, beta blockers, hydralazine, and/or angiotensin receptor-neprilysin inhibitors) can be prepared by any suitable method known to those in the art. For representative methods, see, for example, Francis A. Carey et al., Advanced Organic Chemistry: Part B: Reaction and Synthesis (5^{th} Ed. 2005).

Formulations including a vasodilator (e.g., a calcium channel blocker, ACE inhibitor and/or angiotensin receptor blocker) and at least one excipient are provided. It is generally preferred to administer the compounds of the embodiments in topical formulations; however, other routes of administration, such as intra-articular injection, are also contemplated.

The pharmaceutical compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The pharmaceutical compositions disclosed herein may be manufactured by a process that is itself known, e.g., by means of conventional mixing, dissolving, granulating, dragee-making, levigating, emulsifying, encapsulating, entrapping, tableting, or extracting processes. Many of the vasodilator (e.g., a calcium channel blocker, ACE inhibitor and/or angiotensin receptor blocker) used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intra-articular, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions described herein are suitable for use in treatment or prevention of osteoarthritis syndrome or associated symptoms. The compositions are suitable for use in any patient where treatment or prevention of osteoarthritis syndrome is desirable.

The vasodilator (e.g., a calcium channel blocker, ACE inhibitor and/or angiotensin receptor blocker) can be employed in various types of formulations. Topical formulations including one or more vasodilators in combination with at least one excipient are provided. Excipients can include a nonaqueous or aqueous carrier, and one or more agents selected from moisturizing agents, pH adjusting agents, deodorants, fragrances, chelating agents, preservatives, emulsifiers, thickeners, solubilizing agents, penetration enhancers, anti-irritants, colorants, surfactants, beneficial agents, pharmaceutical agents, and other components as known in the art for use in connection with topical formulations for application to skin. The formulation can be provided as an aqueous formulation, or in an anhydrous formulation which may prevent water-based irritant contact dermatitis or stinging sensation upon application. In another embodiment, the composition is formulated such that preservatives need not be employed (e.g., a preservative-free formulation) so as to avoid skin irritation associated with certain preservatives.

To facilitate application, the composition may be provided as an ointment, an oil, a lotion, a paste, a powder, a gel, or a cream. The composition may also include additional ingredients such as a protective agent, an emollient, a humectant, an antibiotic agent, an antifungal agent, an antiviral agent, an antiprotozoal agent, an anesthetic agent, a steroidal anti-inflammatory agent, a non-steroidal anti-inflammatory agent, an antipruritic agent, an antioxidant agent, an anti-histamine agent, a vitamin or vitamin complex, a hormone, an anti-skin atrophy agent, and combinations thereof. In a further embodiment, the composition may avoid animal or cellular-based materials to avoid irritation. The composition can be applied directly to the skin in the area of the joint or by intra-articular injection.

Methods of using vasodilator formulations are provided. The compositions may be applied topically in the region of the joint, but may also be applied to tissues of the joint directly, e.g., by intra-articular injection.

Some embodiments include administering vasodilator (e.g., a calcium channel blocker, ACE inhibitor and/or angiotensin receptor blocker) compositions provided herein in topical formulations; however, other routes of administration are also contemplated (e.g., intra-articular or the like). Contemplated routes of administration include but are not limited to topical and intra-articular. Suitable liquid forms include suspensions, emulsions, solutions, and the like. Unit dosage forms can also be provided, e.g., individual packets with a premeasured amount of the formulation, configured for administration to the tissue on a predetermined schedule (e.g., daily, weekly, etc.). Unit dosage forms configured for administration twice a day can be employed; however, in certain embodiments it can be desirable to configure the unit dosage form for administration once a day, four times a day, or more, or once every other day, every three days, weekly, or less, or on an as-needed basis.

In some embodiments, the topical and intra-articular formulations typically comprise from about 0.001 wt. % or less to about 50 wt. % or more of active ingredient, such as the vasodilator (e.g., a calcium channel blocker, ACE inhibitor and/or angiotensin receptor blocker), preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Compositions and formulations for topical administration to the tissue of the eye can include gels, drops, sprays, liquids, and aerosols. Conventional pharmaceutical carriers, aqueous or oily bases, thickeners and the like may be employed. Such formulations are typically provided in an eyedropper. A liquid or gel can also be placed using an applicator, e.g., a roller, a stick, a wand, a sponge, a syringe, or other suitable method.

A topical formulation can be provided in a form of a carrier containing the vasodilator, e.g., 50 ppm or less to 1000, 5000, 10000, 50000, 100000, 500000 ppm or more of the vasodilator. The topical formulation can contain from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 10 wt. % or 20 wt. % of the vasodilator. The amount of vasodilator in the base can be adjusted up or down.

Liquids and gels containing the vasodilator, optionally with other components as described herein, can be prepared using techniques as are known in the art for preparing topical compositions. See, e.g., Handbook of Cosmetic Science and Technology, Fourth Edition, edited by André O. Barel, Marc Paye, Howard I. Maibach, CRC Press, 2014, the contents of which is hereby incorporated by reference in its entirety. Various formulations are possible.

For liquid formulations (e.g., gel or lotion forms), a silicone, e.g., a cyclosiloxane or linear silicone (e.g., silicone elastomer), can be employed as a carrier. One type of suitable carrier is a dimethicone crosspolymer gel, e.g., dimethicone crosspolymer in cyclopentasiloxane. Other suitable dimethicone crosspolymers include cyclopentasiloxane, dimethicone/vinyldimethicone crosspolymer; dimethicone, dimethicone/vinyl dimethicone crosspolymer; and isodecane dimethicone/vinyl dimethicone crosspolymer.

Typically, the carrier is present in an amount of from about 80 wt. % to about 95 wt. %, or 82 wt. % to 92 wt. %, e.g., in a topical formulation for application to skin.

Penetration enhancers can be employed to enhance penetration of the vasodilator into tissue. Typical amounts when employed in topical formulations are from 1% by weight to 4% by weight. Typical amounts for anti-irritation agents when employed in topical formulations are from 1% by weight to 4% by weight. Typical amounts for anti-inflammatory agents when employed in topical formulations are from 1% by weight to 4% by weight. Typical amounts for anti-inflammatory agents when employed in topical formulations are from 0.1% by weight to 2% by weight.

In some embodiments, the vasodilator can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration to the tissue of the eye comprise the vasodilator as described herein and a vehicle acceptable for contact with ocular tissue. The vehicle may be aqueous or nonaqueous. The vehicle used in the topical composition may be in the form of a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the vasodilator into the skin tissue or tissue of the joint.

The pharmaceutical excipients used in the topical preparations of the vasodilator compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for hydrophobic topical formulations include mineral oils, vegetable oils, and silicone oils. If desired, the vasodilator compositions as described herein may be dissolved or dispersed in a hydrophobic oil phase, and the oil phase may then be emulsified in an aqueous phase comprising water, alone or in combination with lower alcohols, glycerin, and/or glycols. In certain embodiments water is present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the formulations as described herein can be advantageously employed where it is desirable to minimize osmotic shock.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carragenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

Lecithin and other phospholipids may be used to prepare liposomes containing the vasodilators as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the vasodilators as described herein.

The topical formulation may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The vasodilator compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When the vasodilator formulations of the embodiments are administered by intra-articular injection, it is preferably in the form of a pyrogen-free, parenterally acceptable aqueous solution or oleaginous suspension, emulsion or solution. Suspensions can be formulated according to methods well known in the art using suitable dispersing or wetting agents and suspending agents. The preparation of acceptable aqueous or nonaqueous solutions with suitable properties, e.g., pH, isotonicity, stability, and the like, is within the skill in the art. For example, an isotonic vehicle such as 1,3-butanediol, water, isotonic sodium chloride solution, Ringer's solution, dextrose solution, dextrose and sodium chloride solution, lactated Ringer's solution, or other vehicles as are known in the art can be employed, or a fixed oil can be employed conventionally as a solvent or suspending medium, e.g., synthetic mono or diglycerides, fatty acids, or the like. The vasodilator formulations can also contain stabilizers, preservatives, buffers, antioxidants, or other additives known to those of skill in the art.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

### Kits for Administration of Compositions

Some embodiments of the methods and compositions provided herein include kits comprising vasodilators provided herein. In some embodiments, kits can be provided to an administering physician, other health care professional, a patient, or a caregiver. In some embodiments, a kit comprises a container which contains the vasodilator(s) in a suitable topical formulation, and instructions for administering the composition to a subject. The kit can optionally also contain one or more additional therapeutic or other agents. For example, a kit containing a vasodilator blocker in topical form can be provided along with other agents such as topical antibiotics or topical anesthetics. The kit may contain the vasodilator in bulk form, or can contain separate doses of the vasodilator for serial or sequential administration. The kit can optionally contain one or more diagnostic tools, administration tools, and/or instructions for use, e.g., syringes for intra-articular injection. The kit can contain suitable delivery devices, such as, rollers, sticks, syringes, pump dispensers, wands, single dose packets, and the like, along with instructions for administering the vasodilator compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

The topical formulation for administration to tissue of the joint or the skin adjacent to the joint, in addition to the vasodilator, can contain other ingredients.

While topical administration of the vasodilator disclosed herein can advantageously be employed, in certain embodiments other routes of administration are also contemplated, such as intra-articular injection.

The vasodilator compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The vasodilator compositions disclosed herein may be manufactured into administrable forms by a process that is itself known, e.g., by means of conventional mixing, dissolving, granulating, dragee-making, levigating, emulsifying, encapsulating, entrapping, tableting, or extracting processes.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal, intra-articular, and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the vasodilator may be combined as the active ingredient in intimate admixture with a pharmaceutical carrier according to conventional pharmaceutical compounding techniques. The excipients are preferably minimized so as to ensure administration of an appropriate amount of vasodilator in a compact format. The carrier can take a wide variety of forms depending on the form of preparation desired for administration. Thus, the vasodilator compositions provided herein can be presented as discrete units suitable for administration each containing a predetermined amount of the active ingredient. Further, the vasodilator compositions can be presented as an oil, as a solution, as a suspension in an aqueous liquid, as a non-aqueous liquid, as an oil-in-water emulsion, or as a water-in-oil liquid emulsion, similar to the topical formulations described elsewhere herein, but using components suitable for human contact or consumption. In addition to the common dosage forms set out above, the vasodilator compositions provided herein can also be administered by controlled release and/or delivery devices. The vasodilator compositions can be prepared by any of the methods of pharmacy. In general, such methods include a step of bringing into association the active ingredient with the carrier that constitutes one or more necessary ingredients. In general, the vasodilator compositions are prepared by uniformly and intimately admixing the vasodilator ingredient(s) with liquid carriers or finely divided solid carriers or both. The product can then be conveniently shaped into the desired presentation.

A vasodilator formulation may also be administered in a local manner, for example, via injection of the vasodilator composition directly into a target area, e.g., in a depot or sustained release formulation intra-articularly. Furthermore, a targeted drug delivery system for the vasodilator may be used, for example, in a liposome coated with a tissue specific antibody.

The vasodilator compositions may contain the vasodilator in an amount effective for the desired therapeutic effect. In some embodiments, the vasodilator compositions are in a unit dosage form and comprise from about 0.1 mg or less to about 5000 mg or more of vasodilator per unit dosage form. In further embodiments, the vasodilator compositions comprise from about 1 to about 500 mg per unit dosage form or from about 500 to 5000 mg per unit dosage form of vasodilator. Such amounts can be selected depending upon the vasodilator employed. Such dosage forms may be solid, semisolid, liquid, an emulsion, or adapted for delivery via aerosol or the like.

The carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

Vasodilator compositions provided herein can be prepared as solutions or suspensions of the vasodilator in water or nonaqueous liquids. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Vasodilator compositions provided herein suitable for injectable use include sterile aqueous solutions or dispersions. Furthermore, the vasodilator compositions can be in the form of sterile powders for the extemporaneous preparation of such sterile injectable solutions or dispersions. The vasodilator compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the vasodilator formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood or other bodily fluids of the intended recipient. Vasodilator compositions can also be prepared in powder or liquid concentrate form for dilution.

Contemplated herein are vasodilator compositions including one or more vasodilators as described herein in combination with at least one additional active agent, e.g., an antibiotic. The vasodilator and the at least one additional active agent(s) may be present in a single formulation or in multiple formulations provided together, or may be unformulated. In some embodiments, the vasodilator can be administered with one or more additional agents together in a single composition. For example, the vasodilator can be administered in one composition, and at least one of the additional agents can be administered in a second composition. In a further embodiment, the vasodilator and the at least one additional active agent(s) are co-packaged in a kit. For example, a drug manufacturer, a drug reseller, a physician, a compounding shop, or a pharmacist can provide a kit comprising the vasodilator in combination with another product or component for delivery to a patient. Such additional components can include anti-infective agents, anti-inflammatory agents, anesthetics, or the like.

Some embodiments described herein relate to compositions of vasodilator, which can include a therapeutically effective amount of the vasodilator described herein and a pharmaceutically acceptable carrier, diluent, excipient or combination thereof. The vasodilator composition can include the vasodilator in an amount for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition.

### EXAMPLES

### Example 1

A patient is diagnosed with osteoarthritis syndrome in both knees. A composition comprising the calcium channel blocker nifedipine is directly applied to the skin in the region of one knee while the other knee is left untreated. The treated knee is observed to have reduced symptoms of osteoarthritis syndrome than the untreated knee.

### Example 2

A patient is diagnosed with osteoarthritis syndrome in both knees. A composition comprising the ACE inhibitor enalapril is directly applied to the skin in the region of one knee while the other knee is left untreated. The treated knee is observed to have reduced symptoms of osteoarthritis syndrome than the untreated knee.

### Example 3

A patient is diagnosed with osteoarthritis syndrome in both knees. A composition comprising the angiotensin receptor blocker losartan is directly applied to the skin in the region of one knee while the other knee is left untreated. The treated knee is observed to have reduced symptoms of osteoarthritis syndrome than the untreated knee.

### Example 4

A patient is diagnosed with osteoarthritis syndrome in both knees. A composition comprising the calcium channel blocker nifedipine is administered by intra-articular injection in one knee while the other knee is left untreated. The treated knee is observed to have reduced symptoms of osteoarthritis syndrome than the untreated knee.

### Example 5

A patient is diagnosed with osteoarthritis syndrome in both knees. A composition comprising the ACE inhibitor enalapril is administered by intra-articular injection in one knee while the other knee is left untreated. The treated knee is observed to have reduced symptoms of osteoarthritis syndrome than the untreated knee.

### Example 6

A patient is diagnosed with osteoarthritis syndrome in both knees. A composition comprising the angiotensin receptor blocker losartan is administered by intra-articular injection in one knee while the other knee is left untreated. The treated knee is observed to have reduced symptoms of osteoarthritis syndrome than the untreated knee.

### Exemplary Pharmaceutical Compositions and Methods

Pharmaceutical Composition 1: A pharmaceutical composition for the treatment of osteoarthritis syndrome, comprising: at least one vasodilator; and at least one pharmaceutical excipient.

Pharmaceutical Composition 2: Pharmaceutical Composition 1, for the treatment of osteoarthritis syndrome in the knee.

Pharmaceutical Composition 3: Pharmaceutical Composition 1, for the treatment of osteoarthritis syndrome in a joint other than the knee.

Pharmaceutical Composition 4: Any One of Pharmaceutical Compositions 1 through 3, in a form adapted for topical administration or intra-articular injection to an affected joint.

Pharmaceutical Composition 5: Pharmaceutical Composition 4, wherein the form is selected from the group consisting of an oil, a liquid and a suspension for topical application on the skin in the region of an affected joint.

Pharmaceutical Composition 6: Any One of Pharmaceutical Compositions 1 through 3, formulated as a liquid or a suspension of the at least one vasodilator, wherein the vasodilator is a contact vasodilator.

Pharmaceutical Composition 7: Any One of Pharmaceutical Compositions 1 through 6, wherein the vasodilator is a calcium channel blocker.

Pharmaceutical Composition 8: Pharmaceutical Composition 7, wherein the at least one calcium channel blocker is a dihydropyridine selected from the group consisting of nifedipine, isradipine, felodipine, amlodipine, nicardipine, and clevidipine.

Pharmaceutical Composition 9: Pharmaceutical Composition 7, wherein the at least one calcium channel blocker is a non dihydropyridine selected from the group consisting of verapamil and diltiazem.

Pharmaceutical Composition 10: Any One of Pharmaceutical Compositions 1 through 6, wherein the vasodilator is an ACE inhibitor.

Pharmaceutical Composition 11: Pharmaceutical Composition 10, wherein the ACE inhibitor is selected from the group consisting of benazepril, captopril, enalapril, fosinopril, lisinopril, moexipril, perindopril, quinapril, ramipril, and trandolapril.

Pharmaceutical Composition 12: Any One of Pharmaceutical Compositions 1 through 6, wherein the vasodilator is an angiotensin receptor blocker.

Pharmaceutical Composition 13: Pharmaceutical Composition 12, wherein the angiotensin receptor blocker is selected from the group consisting of azilsartan, candesartan, eprosartan, irbesartan, losartan, olmesartan, telmisartan, and vaisartan.

Pharmaceutical Composition 14: Any One of Pharmaceutical Compositions 1 through 6, wherein the vasodilator is a nitrate.

Pharmaceutical Composition 15: Pharmaceutical Composition 14, wherein the nitrate is selected from the group consisting of nitroglycerin, isosorbide mononitrate and isosorbide dinitrate.

Pharmaceutical Composition 16: Any One of Pharmaceutical Compositions 1 through 6, wherein the vasodilator is an alpha blocker.

Pharmaceutical Composition 17: Pharmaceutical Composition 16, wherein the alpha blocker is selected from the group consisting of doxazosin, prazosin, and terazosin.

Pharmaceutical Composition 18: Any One of Pharmaceutical Compositions 1 through 6, wherein the vasodilator is a beta blocker.

Pharmaceutical Composition 19: Pharmaceutical Composition 18, wherein the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

Pharmaceutical Composition 20: Any One of Pharmaceutical Compositions 1 through 6, wherein the vasodilator is hydralazine.

Pharmaceutical Composition 21: Any One of Pharmaceutical Compositions 1 through 6, wherein the vasodilator is an angiotensin receptor-neprilysin inhibitor.

Pharmaceutical Composition 22: Pharmaceutical Composition 21, wherein the angiotensin receptor-neprilysin inhibitor is sacubitril/valsartan.

Pharmaceutical Composition 23: Any One of Pharmaceutical Compositions 1 through 22, wherein the concentration of the vasodilator is about 0.0001 mg per ml to 1000 mg per ml, optionally 1 mg per ml to 10 mg per ml, optionally 1 mg per ml to 1000 mg per ml, optionally 5 mg per ml to 10 mg per ml, optionally 10 mg per ml, optionally 20 mg per ml, optionally 30 mg per ml, optionally 60 mg per ml, optionally 90 mg per ml, optionally 120 mg per ml, optionally 180 mg per ml, optionally 240 mg per ml.

Pharmaceutical Composition 24: Any One of Pharmaceutical Compositions 1 through 2, wherein the concentration of the vasodilator is from about 0.0001% by weight to about 20% by weight, optionally about 0.01% by weight, optionally about 0.1% by weight, optionally about 1% by weight, optionally about 10% by weight, optionally about 20% by weight.

Method 25: A method for the treatment of osteoarthritis syndrome in a patient in need thereof, comprising: administering an effective amount of the pharmaceutical composition according to any one of Pharmaceutical Compositions 1 through 24 to a patient in need thereof.

Method 26: Method 25, for the treatment or prophylaxis of osteoarthritis syndrome in the knee.

Method 27: Method 25, for the treatment or prophylaxis of osteoarthritis syndrome in a joint other than a knee.

Method 28: Method 25, wherein the composition is administered once a day, optionally two or more times a day, optionally once a week, optionally two or more times a week, optionally once a month, optionally two or more times a month, optionally a plurality of times a year.

Any of the features the above referenced pharmaceutical compositions, uses, and methods is applicable to any other pharmaceutical composition, use, or method identified herein. Moreover, any of the features of the above referenced pharmaceutical compositions, uses, and methods is independently combinable, partly or wholly, with other embodiments of the pharmaceutical compositions, uses, and methods described herein in any way, e.g., one, two, or three or more features may be combinable in whole or in part. Further, any of the features of the pharmaceutical compositions, uses, and methods described above may be made optional to other pharmaceutical compositions, uses, and methods described herein. Any aspect or embodiment of a method or use described herein can be performed using a composition, e.g., a pharmaceutical composition and/or a compound as described herein, and any aspect or embodiment of a composition, e.g., a pharmaceutical composition and/or a compound described herein, can be used or adapted to perform a method or use as described herein.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A pharmaceutical composition for the treatment of osteoarthritis syndrome, comprising:
at least one vasodilator; and
at least one pharmaceutical excipient.

2. The pharmaceutical composition of claim 1, wherein the pharmaceutical composition is in a form adapted for topical administration or intra-articular injection to an affected joint.

3. The pharmaceutical composition of claim 2, wherein the form is selected from the group consisting of an oil, a liquid and a suspension for topical application on the skin in the region of an affected joint.

4. The pharmaceutical composition claim 1, wherein the pharmaceutical composition is formulated as a liquid or a suspension of the at least one vasodilator, wherein the vasodilator is a contact vasodilator.

5. The pharmaceutical composition of claim 1, wherein the vasodilator is a calcium channel blocker.

6. The pharmaceutical composition of claim 5, wherein the at least one calcium channel blocker is a dihydropyridine selected from the group consisting of nifedipine, isradipine, felodipine, amlodipine, nicardipine, and clevidipine.

7. The pharmaceutical composition of claim 5, wherein the at least one calcium channel blocker is a non dihydropyridine selected from the group consisting of verapamil and diltiazem.

8. The pharmaceutical composition of claim 1, wherein the vasodilator is an ACE inhibitor selected from the group consisting of benazepril, captopril, enalapril, fosinopril, lisinopril, moexipril, perindopril, quinapril, ramipril, and trandolapril.

9. The pharmaceutical composition of claim 1, wherein the vasodilator is an angiotensin receptor blocker selected from the group consisting of azilsartan, candesartan, eprosartan, irbesartan, losartan, olmesartan, telmisartan, and valsartan.

10. The pharmaceutical composition of claim 1, wherein the vasodilator is a nitrate selected from the group consisting of nitroglycerin, isosorbide mononitrate and isosorbide dinitrate.

11. The pharmaceutical composition of claim 1, wherein the vasodilator is an alpha blocker selected from the group consisting of doxazosin, prazosin, and terazosin.

12. The pharmaceutical composition of claim 1, wherein the vasodilator is a beta blocker selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

13. The pharmaceutical composition of claim 1, wherein the vasodilator is hydralazine.

14. The pharmaceutical composition of claim 1, wherein the vasodilator is an angiotensin receptor-neprilysin inhibitor.

15. The pharmaceutical composition of claim 14, wherein the angiotensin receptor-neprilysin inhibitor is sacubitril/valsartan.

16. The pharmaceutical composition of claim 1, wherein the concentration of the vasodilator is about 0.0001 mg per ml to 1000 mg per ml, optionally 1 mg per ml to 10 mg per ml, optionally 1 mg per ml to 1000 mg per ml, optionally 5 mg per ml to 10 mg per ml, optionally 10 mg per ml, optionally 20 mg per ml, optionally 30 mg per ml, optionally 60 mg per ml, optionally 90 mg per ml, optionally 120 mg per ml, optionally 180 mg per ml, optionally 240 mg per ml.

17. A method for the treatment of osteoarthritis syndrome in a patient in need thereof, comprising:
administering an effective amount of the pharmaceutical composition according to claim 1 to a patient in need thereof.

18. The method of claim 17, for the treatment or prophylaxis of osteoarthritis syndrome in a knee.

19. The method of claim 17, for the treatment or prophylaxis of osteoarthritis syndrome in a joint other than a knee.

20. The method of claim 17, wherein the composition is administered once a day, optionally two or more times a day, optionally once a week, optionally two or more times a week, optionally once a month, optionally two or more times a month, optionally a plurality of times a year.
